(19) United States
(12) Patent Application Publication
(10) Pub. No.: US 2016/0103052 A1
(43) Pub. Date: Apr. 14, 2016

(54) SHEAR TESTING DEVICE AND METHOD

(71) Applicant: CLEMSON UNIVERSITY, Clemson, SC (US)

(72) Inventors: Betiglu Eshete Jimma, Clemson, SC (US); Prasada Rangraju, Clemson, SC (US)

(21) Appl. No.: 14/878,534

(22) Filed: Oct. 8, 2015

Related U.S. Application Data
(60) Provisional application No. 62/061,454, filed on Oct. 8, 2014.

(51) Int. Cl. G01N 11/04; G01N 33/38

(52) U.S. Cl. CPC G01N 11/04; G01N 33/383

(57) ABSTRACT

A shear testing device and methods for using the device are described. The device can be portable and can quickly and effectively characterize the workability of a granular material such as a fresh pervious concrete. The device can offer a variety of advantages as a workability-characterizing device: it can be small in size, lightweight and can include a simple, gravity-based working mechanism that makes it a suitable device for both field and lab applications.

### CROSS REFERENCE TO RELATED APPLICATION

The present application claims filing benefit of U.S. Provisional Patent Application Ser. No. 62/061,454, entitled “Shear Testing Device and Method”, confirmation number 5317, having a filing date of Oct. 8, 2014, which is incorporated herein by reference in its entirety.

### BACKGROUND

Granular materials such as concrete present unique difficulties in processing. For instance, Portland cement pervious concrete (PCPC), which typically contains coarse aggregates (50-65 vol. %), paste (15-30 vol. % (water, cement, and any additional non-aggregate additives)), and air voids (15-35 vol. %) exhibits unique properties both in the fresh and hardened state. These unique properties present workability issues associated with PCPC as do similar issues for other granular materials such as ceramic mixtures, granular powders, foods, slurries, and the like. Some of these workability issues include difficulty in mixing (e.g., long PCPC mixing time), difficulty of flow (e.g., through a pipe or chute), prevention of discharge or plugs (e.g., a mixer discharge, a heater discharge, etc.), difficulties in distribution (e.g., even spreading on a rack or conveyor, placing and in finishing of concrete), etc.

To address these workability issues during mixture formulation stage as well as following production, it is necessary to accurately assess the characteristic parameters (workability properties) of the granular material, which requires adequate characterization tools. For instance, the slump test, the most common concrete workability test, fails to provide meaningful information on workability of pervious concrete due to its unique character. As such, visual inspection is the widely practiced test of workability in the pervious concrete industry. Unfortunately, while visual inspection is a reliable technique for experienced professionals, it does not provide a measureable metric that can be used among different parties such as engineers, architects, contractors and producers.

A plurality of testing devices and methods have been developed that can provide a plethora of information about granular materials including PCPC. Unfortunately, most of these devices are quite expensive, often large, rely on a power source, and are useful only in controlled laboratory settings.

Accordingly, what is needed in the art is a device and method that can be used for determining characteristic parameters of granular materials. More specifically, what is needed is an inexpensive device that can be utilized in the field with little preparation or training and that can accurately determine shear strength parameters, which are correlated with workability properties. The use of such a device can quickly and easily provides information regarding, for example, the spreadability, flowability, compactability of the granular material such as pervious concrete, e.g., PCPC.

### SUMMARY

According to one embodiment, disclosed is a shear testing device. In general, a device can include a base, a sample container, and a hoist. The base includes a containment frame and defines a receiving area. The containment frame defines a first opening at an upper surface of the containment frame. The base also has a first end that is closer to the containment frame than to the receiving area.

The sample container defines a second opening that is at a lower surface of the sample container. The sample container is slidably mountable on the base such that at a first position, the first opening and the second opening are aligned and form a shear plane at an interface of the containment frame and the sample container.

The hoist is in mechanical communication with the first end of the base and can be utilized to lift and lower the first end.

Also disclosed is a method for determining one or more shear strength parameters of a granular material. A method can include aligning a first opening of a containment frame of a base of a shear testing device with a second opening of a sample container such that the containment frame and the sample container together define a sample loading area and define a shear plane at an interface of the containment frame and the sample container. During the method, the sample container can be slidably mounted on the base.

The method also includes loading a granular material sample into the sample loading area and lifting a first end of the base. The first end is raised to a lift point at which the sample container slides from a first position to a second position due to gravitational force acting on the sample container. At the lift point, the base is at an angle to horizontal. The one or more shear strength parameters of the granular material can then be determined from this angle.

### BRIEF DESCRIPTION OF THE DRAWINGS

A full and enabling disclosure, including the best mode thereof, to one of ordinary skill in the art, is set forth more particularly in the remainder of the specification, including reference to the accompanying Figures, in which:

FIG. 1 is a perspective view of a shear testing device assembly.

FIG. 2 is another perspective view of a shear testing device assembly.

FIG. 3 is another view perspective view of a shear testing device assembly.

FIG. 4 is a side view of a shear testing device assembly.

FIG. 5 is a perspective view of a base frame.

FIG. 6 is a top view of a base frame.

FIG. 7 is a perspective view of a containment frame.

FIG. 8 is another perspective view of a containment frame.

FIG. 9 is a top view of a containment frame.

FIG. 10 is a perspective view of a sample container.

FIG. 11 is a top view of a sample container.

FIG. 12 is another perspective view of a sample container.

FIG. 13 is a side view of a support.

FIG. 14 is a side view of a fully assembled shear testing device.

FIG. 15 is a perspective view of a fully assembled shear testing device.

FIG. 16 is a front view of a fully assembled shear testing device.

FIG. 17 is a rear view of a fully assembled shear testing device.

FIG. 18 is a schematic illustration of a shear analysis test as may be carried out by use of the disclosed devices.

FIG. 19 illustrates the relationship between shear stress and normal stress.

FIG. 20 is a schematic illustration of the use of a concrete rake to pull a sample.

FIG. 21 presents the relationship between the shear stress and the normal stress obtained for several samples according to the disclosed methods.

FIG. 22A presents the relationship between normal stress and shear stress as determined by a previously known shear device. [0033a] FIG. 22B presents the relationship between normal stress and shear stress as determined by a device as disclosed.

FIG. 23 presents the relationship between normal stress and shear stress as determined as described herein.

FIG. 24 illustrates an isoresponse surface of the coefficient of internal friction of PCPC mixtures proportioned for specific volumetric ratios of voids, paste, and aggregate.

FIG. 25 illustrates an isoresponse surface of internal cohesion for PCPC mixtures proportioned for specific volumetric ratios of voids, paste and aggregate.

FIG. 26 illustrates an isoresponse surface of rake pulling force required for spreading 50 mm thick fresh PCPC mixture proportioned for specific volumetric ratios of voids, paste, and aggregate.

Repeat use of reference characters in the present specification and drawings is intended to represent the same or analogous features or elements of the present disclosure.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment may be used in another embodiment to yield a still further embodiment. Thus, it is intended that the present disclosure cover such modifications and variations as come within the scope of the appended claims and their equivalents.

The present disclosure is generally directed to a shear testing device and methods for using the device. The device can be portable and can quickly and effectively characterize the workability of a granular material such as a fresh pervious concrete. (The terms “pervious concrete,” “Portland Cement perveous concrete,” and “PCPC” are used synonymously and interchangeably throughout this disclosure.) The device can offer a variety of advantages as a workability-characterizing device: it can be small in size, lightweight and can include a simple, gravity-based working mechanism that makes it a suitable device for both field and lab applications.

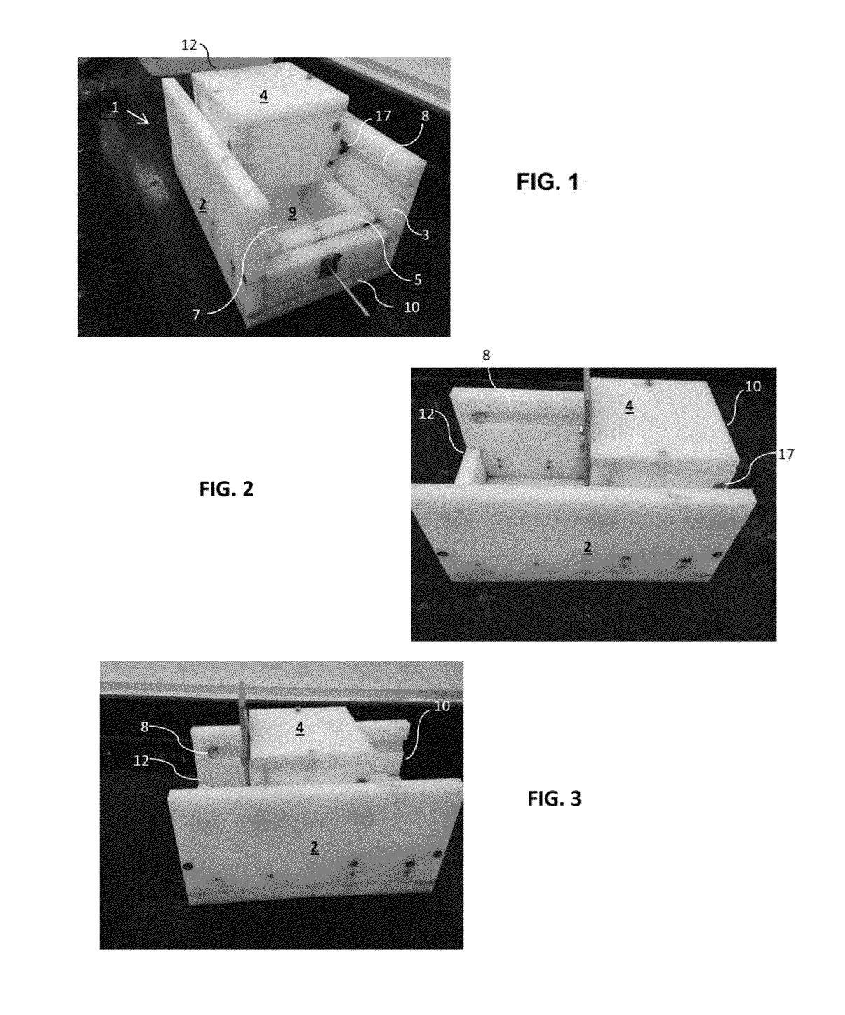

FIG. 1 is a perspective view of one embodiment of an assembly 1 of a shear testing device. In general, the assembly 1 can include a base 2 and a sample container 4. FIG. 2 is another perspective view of the assembly 1 and FIG. 3 is a side view of the assembly 1.

The sample container 4 can be slidably mounted on the base 2 such that it can slide from a first end 10 of the base 2 to a second end 12 of the base 2. At a first position, the sample container 4 can be aligned with a first end 10 of the base 2, and upon sliding to a second position as illustrated in FIG. 3 the sample container can be closer to the second end 12 of the base.

The slidable mounting can utilize any suitable material or device. For instance, in the illustrated embodiment the sample container 4 can be mounted to the base 2 by use of multiple frictionless bearings 17 that fit with a track 8 of the base. The components of a frictionless bearing can be formed of low friction materials as are known in the art such as stainless steel and/or low friction thermoplastic materials including, without limitation, polyamides (e.g., nylons), high density polyethylene (HDPE), polytetrafluoroethylene (PTFE), and so forth.

The base 2 and sample container 4 can be formed of polymeric or other materials that can be successfully utilized under the expected testing conditions. For instance, the base 2 and sample container 4 can be formed of thermoplastic materials that can be quickly and easily cleaned following use and can hold up over long term use in the field.

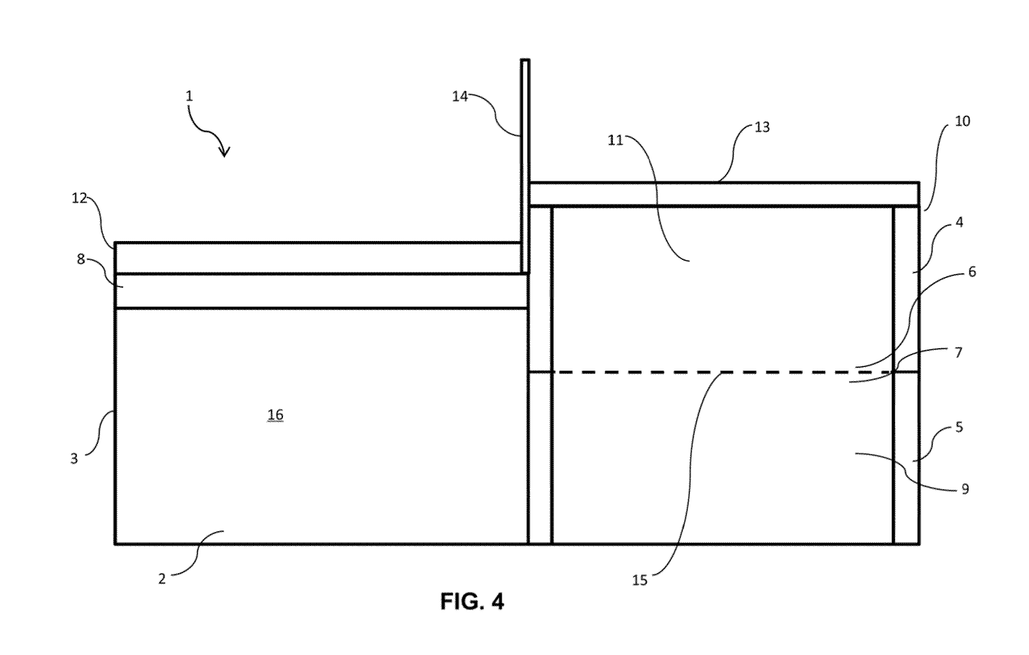

The base 2 can include a base frame 3 and a containment frame 5. The containment frame 5 fits within the base frame 3 as illustrated in FIG. 1 and FIG. 4. As shown in the cutaway side view of FIG. 4, the containment frame 5 can be closer to the first end 10 of the base than to the second end 12 of the base. The base also defines a receiving area 16 that is closer to the second end 12 of the base 2 as compared to the containment frame 5. The containment frame 5 can be removably attachable to the base frame 3 or can be a permanent feature of a monolithic base, as desired.

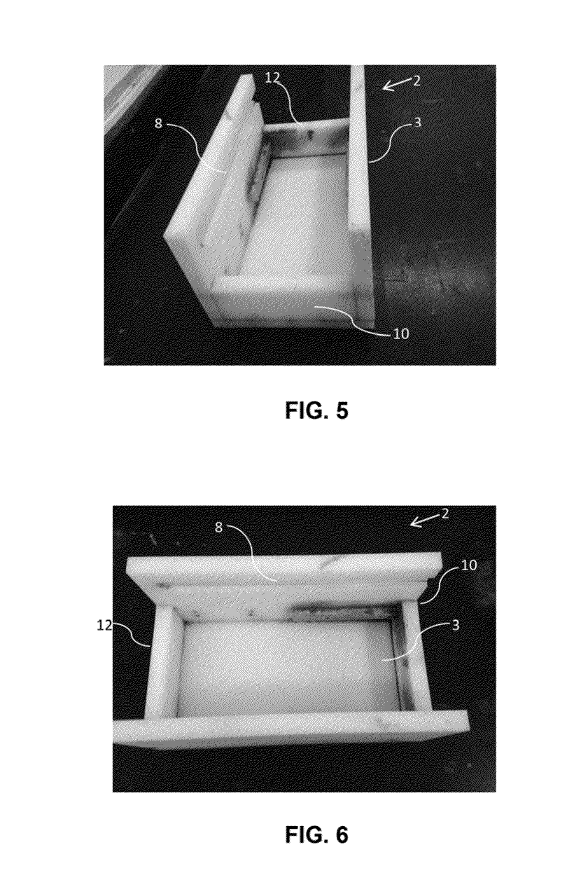

The base 2 including base frame 3 is further illustrated in FIG. 5 and FIG. 6, which show the base frame 3, the track 8 for slidably mounting the sample container 4 with the base 2 and the first end 10 and the second end 12 of the base 2. Of course, the base frame 3 can include a track 8 or other suitable feature for slidably mounting the sample container on both sides of the frame.

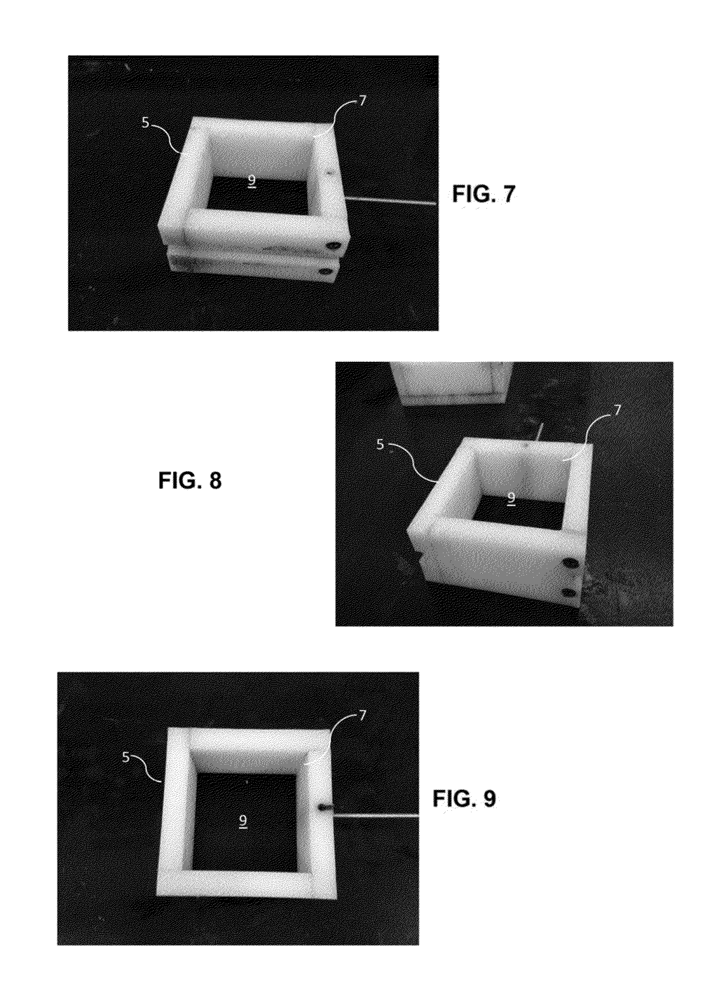

The base 2 also includes a containment frame 5, a separable version of which is illustrated in FIG. 7 in a perspective view, FIG. 8 in another perspective view, and FIG. 9 in a top view. The containment frame 5 fits inside the base frame 3. The fit between the containment frame 5 and the base frame 3 can be seen in FIG. 1 and in FIG. 4 in which the base 2 and the sample container 4 are assembled. As can be seen, the containment frame 5 can be open at the top and bottom surface. Thus, upon assembly of the containment frame 5 and the base frame 3 (or upon formation of a monolithic base) the containment frame 5 can define a first opening 7 at the upper surface of the containment frame 5. In addition, the containment frame 5 when assembled with the base frame 3 can define a containment area 9 within the walls of the containment frame 5. This containment area 9 can serve to hold a portion of a sample to be tested in the device, as explained in more detail below.

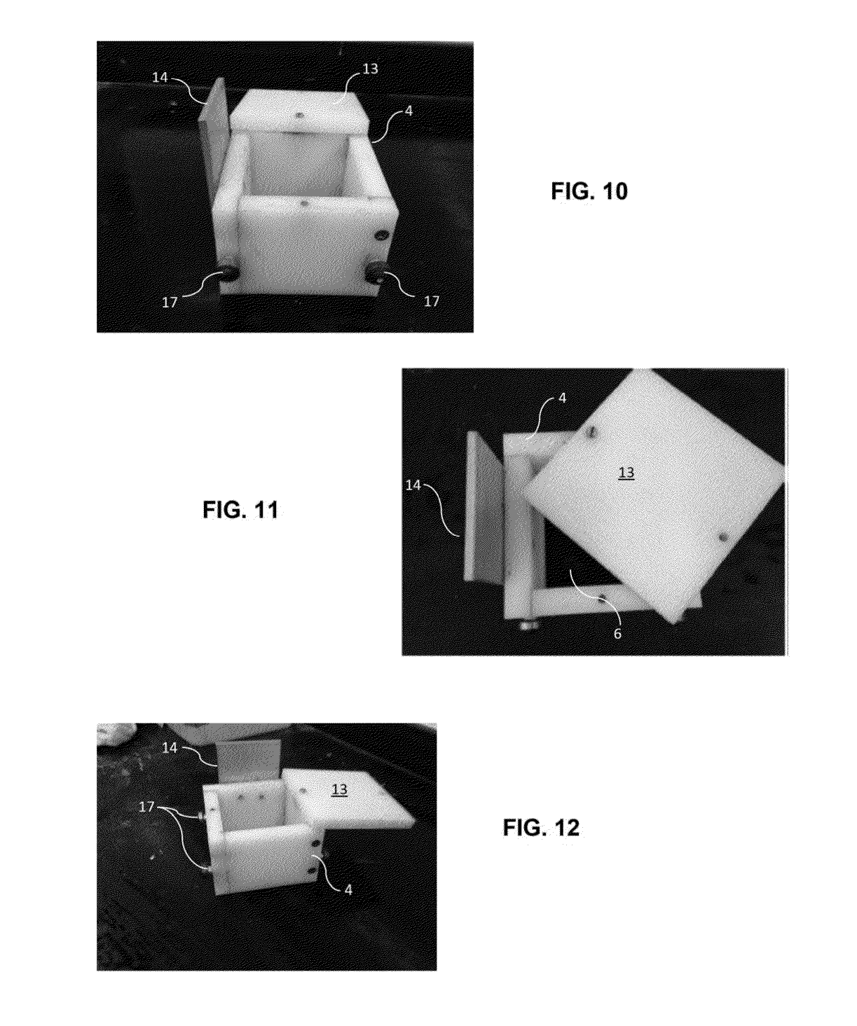

A sample container 4 that can be slidably mounted with the base 2 is illustrated in more detail in FIG. 10, a top view in FIG. 11, and a perspective view in FIG. 12. The sample container 4 includes frictionless bearings 17 for slidably mounting the sample container 4 in the base 2. In addition, the sample container includes a lid 13 and a receiver 14. The lid 13 and receiver 14 can be utilized to add load to the device during a testing procedure, as discussed further below.

As can be seen in FIG. 11, the sample container 4 includes an opening 6 at the lower surface. Referring again to FIG. 4, upon assembly of the sample container 4 with the base 2, the sample container 4 can be aligned with the containment frame 5 at the first end 10 of the assembly 1 such that the opening 7 at the top surface of the containment frame 5 and the opening 6 at the lower surface of the sample container 4 are aligned. Upon this alignment, a sample loading area 11 is defined that encompasses the containment area 9 of the containment frame 5 and also the interior of the sample container 4. In addition, the alignment of the sample container 4 with the containment frame 5 serves to define a shear plane 15 at the interface of the containment frame 5 and the sample container 4.

The sample loading area can generally define a volume that can encompass a representative sample size of the granular material to be tested, and is not particularly limited. The testing device can be easily portable in one embodiment, and as such not sized to be unwieldy. In such an embodiment, the sample loading area can be, for instance, from about 500 cubic centimeters (cm³) to about 10,000 cm³, or from about 1,000 cm³ to about 5,000 cm³.

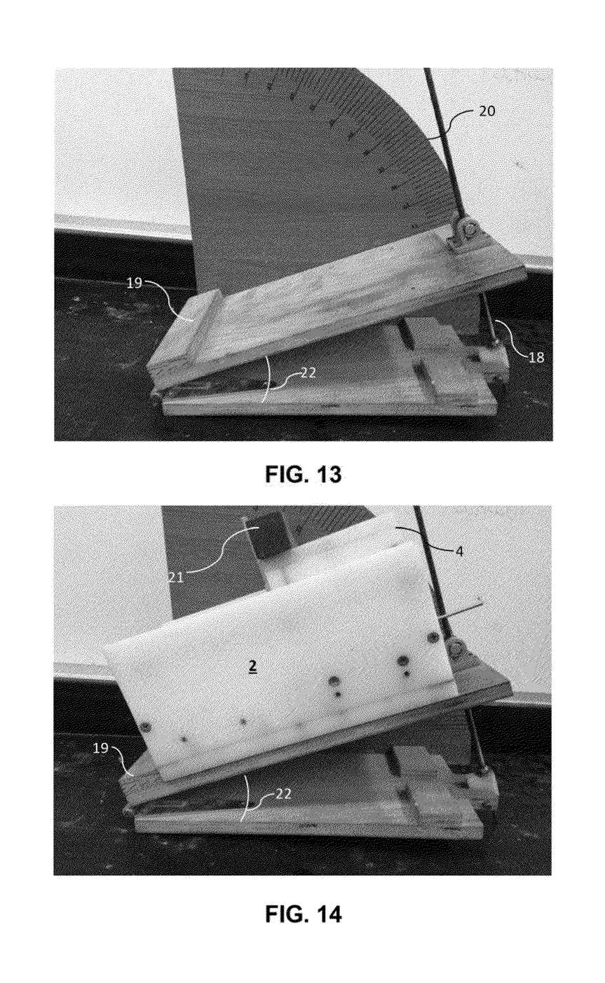

During use, the assembly including the base 2 and the sample container 4 can be held on a support. One embodiment of a support 19 is illustrated in FIG. 13. The support 19 includes a hoist 18 and a lift point indicator 20. The support can be formed of wood (as illustrated), metal, plastic, combinations of materials, etc. as would be evident to one of skill in the art.

The hoist 18 is devised so as to lift one end of the assembly 1. The hoist 18 can be any suitable device, for instance a screw hoist 18 as indicated in FIG. 13, a scissor-type hoist, a ball screw type hoist, telescopic hydraulic hoist, etc. In addition, the hoist can be powered or can be hand-operated. In one embodiment, the hoist can be variably utilized in either a powered or a hand-operated fashion, such that the hoist is useful in any environment (e.g., whether or not an electric power source is available).

During use, the hoist 18 will lift one end of the base/sample container assembly 1 to a lift point at which the sample container 4 will slide across the base 2. The lift point indicator 20 is devised to provide an accurate measure of that lift point. For instance, in the illustrated embodiment, the lift point indicator 20 can provide the angle measurement 22 at the lift point, i.e., the angle from horizontal of the base/sample container at the lift point where the sample container 4 slides away from the containment frame 5 and away from the first end 10 of the base 2. This angle measurement 22 can then be utilized to determine a variety of shear strength parameters.

In other embodiments, the lift point indicator may provide merely the height at which the lift point occurs. For instance, the lift point indicator may be in mechanical communication with the hoist to provide an indication of the hoist height at the lift point. In this embodiment, it is a matter of simple geometry to determine the angle measurement at the lift point.

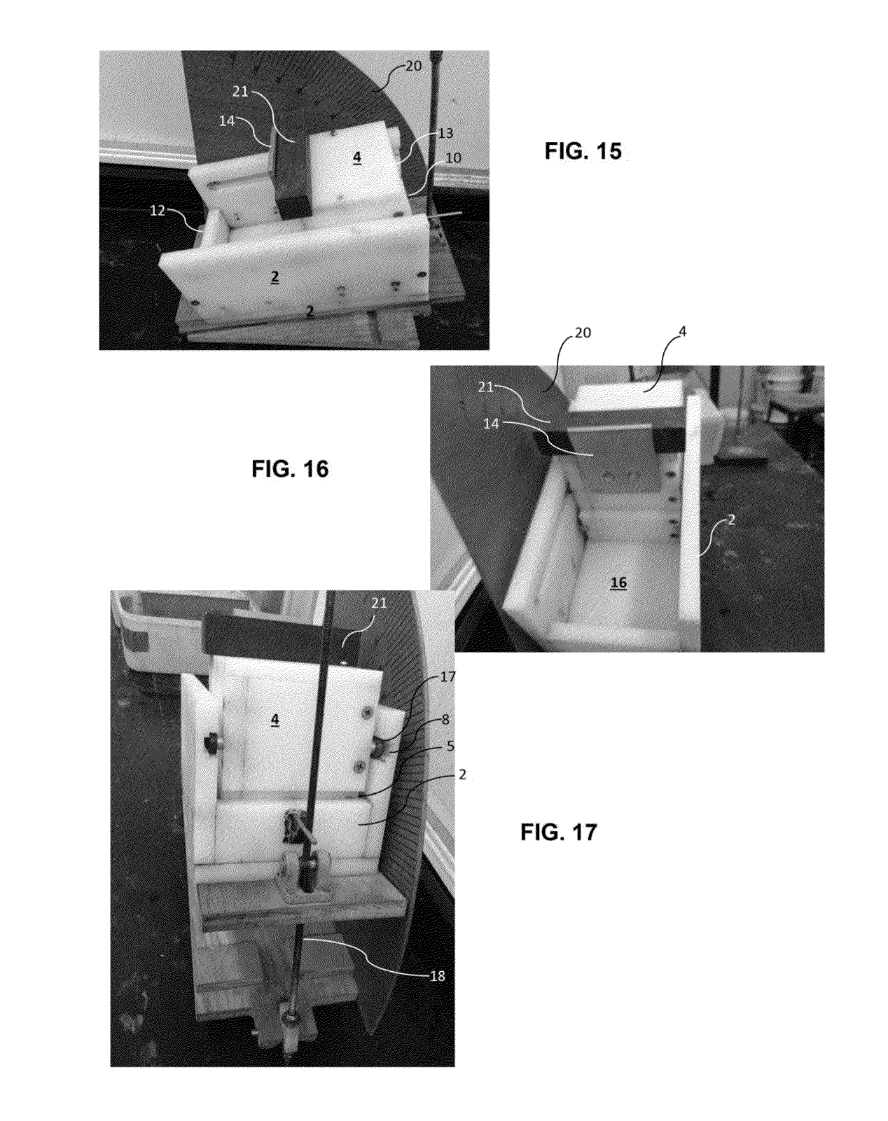

FIGS. 14, 15, 16, and 17 illustrate the fully assembled device. As can be seen, the base 2 can be assembled with the sample container 4 on the support 19 such that the sample container is aligned with the containment frame 5 as shown in FIG. 4. The aligned components together form the sample loading area, as discussed above. A sample of granular material can then be loaded into this sample loading area (reference character 11 on FIG. 4) and the lid 13 closed to cover the sample loading area. The sample size can vary, generally depending upon the specific material to be examined. For instance, when considering concrete, and in one embodiment pervious concrete, the sample size can generally be about 10 kilograms or less, for instance about 5 kilograms or less or about 1 kilogram or less in some embodiments. For example, a PCPC sample of from about 0.5 kilograms to about 3 kilograms can be loaded into the sample loading area formed by the alignment of the containment frame 5 and the sample container 4.

An additional ballast load 21 can be applied to the device during a test run. For instance, an additional ballast load 21 can be held on to the device by use of a receiver 14. A load 21 can be utilized when carrying out multiple runs for a single granular material. For instance, a first run can be carried out with no extra load, a second run can be carried out with an additional ballast load 21 of, e.g., 1 kilogram, and a third run can be carried out with an additional ballast load 21 of, e.g., 5 kilograms. This can provide increased accuracy in the final strength parameters as determined by use of the device. The additional load can vary depending upon the weight of the sample to be tested by the device. For instance, when examining a sample size of about 1 kilogram, the additional load 12 can be from about 1 kilogram to about 10 kilograms, or from about 3 kilograms to about 6 kilograms in one embodiment.

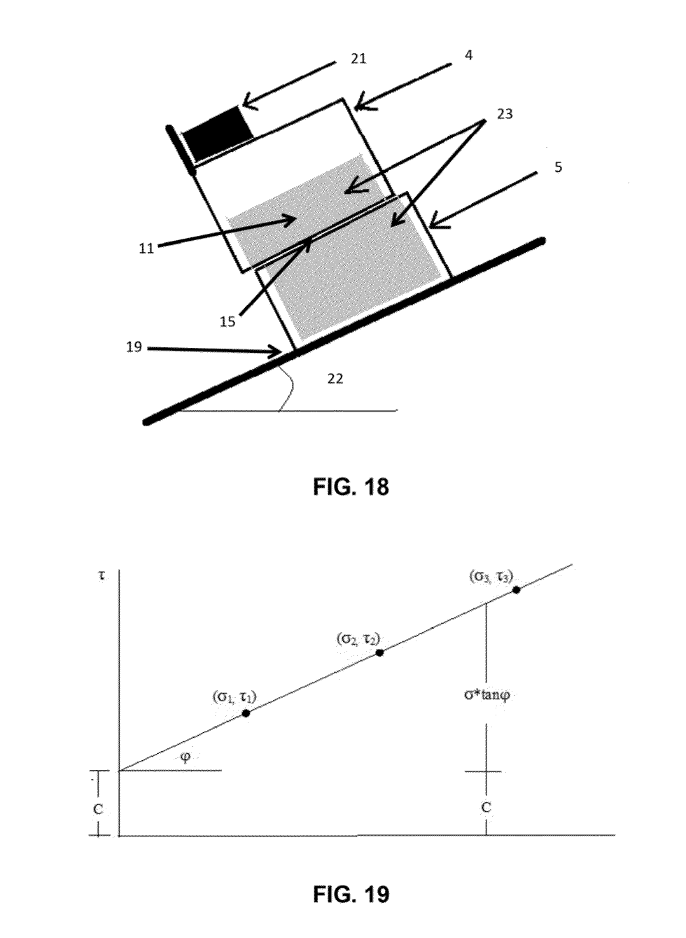

To utilize the device, the hoist is raised to the lift point, at which point the sample container 4 will begin to slide away from the first end 10 of the base toward the opposite end 12 of the device. FIG. 18 is a schematic model of a portion of a device during a test. As shown, a sample of a granular material 23 is contained in a sample loading area 11 formed by the combination of the containment frame 5 and the sample container 4. As the support 19 is lifted to the lift point at the shear angle 22, the sample container 4 slides off of the containment frame 5 due to gravitational force acing along the shear plane 15. Beneficially, it is only the force due to gravity that causes the sample to shear along the shear plane 15, and as such, no externally driven machinery or testing components are required to utilize the shear testing device.

As the sample container 4 slides across the base and off of the upper surface of the containment frame 5, the sample material 23 that was held within the sample container 4 will fall out of the opening in the lower surface of the sample container and can be gathered in the receiving area 16 (FIG. 16). Determination of the amount (weight and/or volume) of this sample material can be utilized in determining shear characteristics of the granular material.

The device can be utilized to characterize the workability of a granular material both quantitatively and qualitatively. A qualitative test can be carried out as described above; by measuring the angle at which the sample container slides relative to the containment frame. This angle (i.e., the shear angle) can be used as a parameter for comparing different granular materials, e.g., different batches of a pervious concrete. In general, a granular material exhibiting a smaller shear angle will be more workable (e.g., more easily spread and compacted) as compared to a granular material exhibiting a larger shear angle. The qualitative test can also be utilized to determine the similarity of two different batches of a granular material. For instance to compare different truckloads of a pervious concrete to insure substantial equality in workability between the different loads. Such a qualitative test is simple and any technician can perform it after few minutes of training.

Quantitative tests can be utilized to determine the shear resistance of the granular material to be tested. This can be used for various purposes, for instance in evaluating the spreadability of a pervious concrete. Spreadability represents the ease with which a mix can be placed and spread to a finishable surface. This property is an important workability property, and often affects the productivity of the finishing crew as well as the quality of the finished concrete pavement surface.

In one embodiment, the disclosed devices can be utilized to evaluate the internal shear resistance of a granular mixture. The shear resistance is a function of the normal stress, the coefficient of internal friction, and the force of cohesion. The normal stress is directly proportional to the self-weight of the material sample held above the shear plane.

FIG. 19 illustrates the general relationship between normal stress and shear stress. This relationship can be described using the following known equation:

τ=C+σ*tan φ

In which:

C=Cohesion

τ=shear stress at failure

σ=Normal stress at failure

tan φ=Coefficient of internal friction

φ=the slope of the line

One embodiment of a method for determination of shear properties can utilize one or more data points obtained by separate runs of the device, for instance separate runs utilizing different additional loads, as discussed above. Similarly, the shear properties can be determined by testing samples with variable sample mass; the larger the sample the higher will be the normal and shear stress at the failure plane.

The shear and normal stress at each data point can be calculated using the following equations:

Normal stress(σ)=(M*9.81*cos η)/A

Shear stress(τ)=[(L+B+M)*9.81*sin Θ]/A

In which:

M=Mass of the sample in the sample container.

L=Additional load

B=Mass of the sample container

Θ=the sample shear angle in degree

A=the surface of the shear plane

The shear strength parameters can be used to compute the pulling force required to spread granular materials such as pervious concrete. For instance, the construction of pervious concrete pavement often involves placing and spreading of the PCPC mixture, therefore spreadability is an important workability property for PCPC. The first spreading activity is often done manually using a rake or wooden board. The force required to pull the rake depends on the workability of the mix. Workable mixes require less raking force; whereas harsh mixes require a lot of force, and can be strenuous on the workers. The total force a finisher applies to pull the rake depends on the amount of material held by the rake, the angle of the rake, and the internal shear resistance of the mixture.

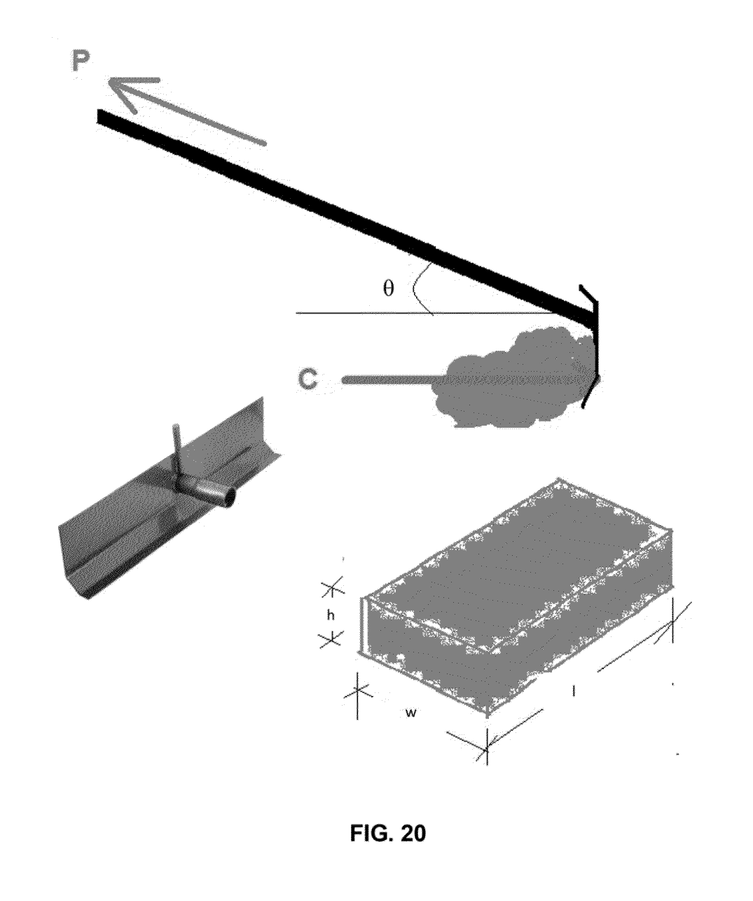

One example of a method for determining the pulling force of a sample can include estimation of a mass of material being pulled as a rectangular block with given dimensions, e.g., width (w)×length (l)×height (h). FIG. 20 schematically illustrates the pulling force required to spread the sample (s).

The pulling force P required can be calculated as follows:

P=R/Cos θ

R=τ*A

τ=C+μ*Fₙ

Fₙ=γ*h

In which:

P=the pulling force

R=is the total resistance from the sample

τ=shear stress of the sample

A=The area of shear

γ=Density of the concrete

h=the height of sample rectangle estimate

Fₙ=Normal stress from self-weight of concrete (σ in the previous calculation)

C=Cohesion

μ=coefficient of internal friction (tan φ in the previous calculation)

The coefficient of internal friction, the shear stress, the cohesion, and the normal stress can all be determined as discussed above.

Shear strength parameters can also be utilized to determine the compaction characteristics of a granular material. For example, PCPC is usually compacted using a roller screed. The compaction and finishing efforts also involve some form of pushing and spreading. Usually, during the rolling process, the roller pushes the material, which is cut off from the finished pavement surface. The material that accumulates in front of the roller creates a resistance force against the progress of the roller. The magnitude of this resistance force is equivalent to the total internal shear stress at the interface of the concrete mass and the pavement surface. The resistance force from the concrete also determines the speed of paving, and the force required to pull the roller. By use of a device as described herein, the shear resistance of a granular material such as concrete can be calculated using a similar procedure as the determination of the pulling force described above.

The present disclosure may be further understood with reference to the example, below.

### Example 1

A device as illustrated in FIG. 14 was utilized. The sample container had an outer dimension of 14.5 cm×14.5 cm×9.5 cm [5.7 in.×5.7 in.×3.7 in.] and the thickness of the wall was 19 mm [0.75 in.]. The containment frame had dimensions of 14.5 cm 14.5 cm×7 cm. The sample container was supported by four frictionless bearings, which slid in two tracks provided on the inside of the base frame. The base fame had outer dimensions of 35 cm×20 cm×17 cm [13.8 in.×8 in.×6.7 in.].

The sample container was held in place on and aligned with the containment frame using a pin-lock provided in the containment frame.

The test procedure for determining the internal shear resistance of a PCPC mix was as follows:

1. The device was placed on a scale and the scale was set to zero.

2. 1600 g of PCPC mix was loaded into the box sample loading area from a height of 200 mm.

3. The device containing the PCPC sample was placed on the support. The angle of the support was positioned to 5°.

4. A 6 kg steel bar was placed on the lid of the sample container as a ballast load to create a shearing force.

5. The pin that held the sample container and the containment frame together was unlocked, and the support was gradually lifted with a constant radial speed of 20 degrees per minute. The lifting of the support was stopped when the sample container displaced relative to the containment frame by more than 30 mm.

6. The angle and the weight of the PCPC mix displaced in the sample container were recorded. These two values were used to calculate the normal and the shearing stresses at failure as described above.

The experiment was repeated two additional times with PCPC sample weights of 2100 g and 2600 g. The sample mass was varied to create different normal and shear forces in the sample.

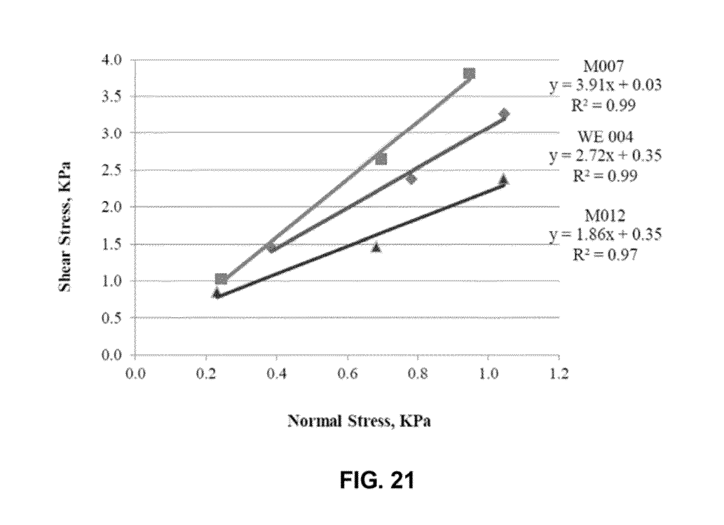

FIG. 21 presents the plot of normal stress vs. shear stress obtained for three selected PCPC mixes. The regression equations are also provided with the plot.

To evaluate the reliability of the measurements obtained from the device and to compute the error in the test method, a repeatability test was conducted on plane aggregates. The repeatability test involves performing 15 repeated measurements on 9.5 mm [3/8 in] size aggregates. The data collected from this test is as shown in the table below. The mass of aggregate used for this test was 2.0 kg. The additional load used to induce gravitational shear force was 2.4 kg. In the test, the coefficient of variation (COV) of the test was slightly high. However, we can conclude that the test was acceptable because the COV was within the acceptable range (<15%).

Measured 17, 28, 19, 17, 22, 18, 17, 19, 17.5, 16.3,
Shear angle 16.5, 20, 21.8, 23, 20

| | |
|---|---|
| Mean shear angle | 18.86 |
| Std Dev | 2.21 |
| Std Err Mean | 0.59 |
| Upper 95% Mean | 20.13 |
| Lower 95% Mean | 17.59 |
| Coefficient of variation (%) | 11.72 |

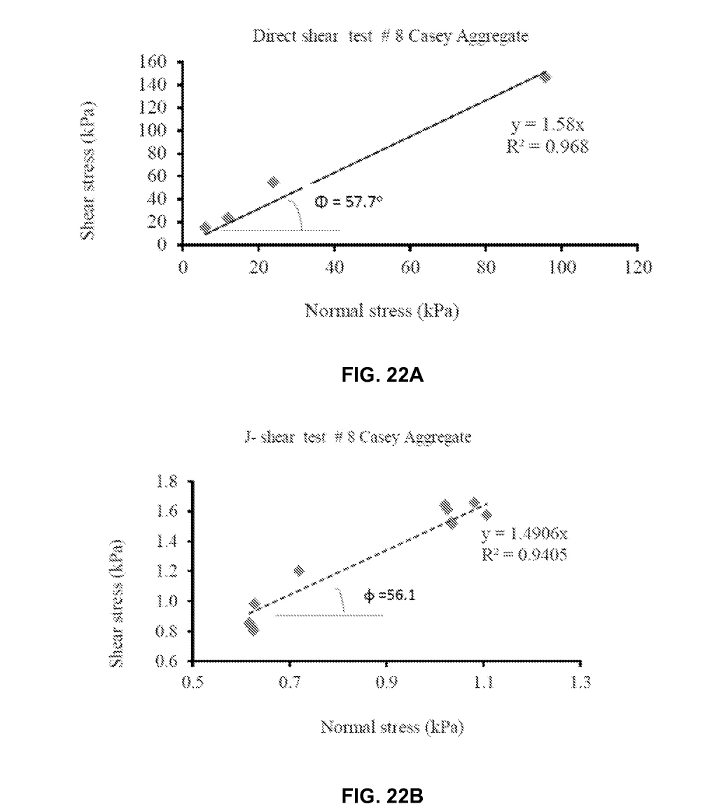

The accuracy of the device was evaluated by comparing the shear parameters obtained according to the disclosed methods with shear parameters determined using the standard direct shear test (ASTM D3080). One size of aggregates 2.36 mm [#8] was tested for this purpose. FIG. 22A illustrates the shear strength parameters obtained by using the direct shear test and FIG. 22B illustrates the parameters obtained by using a disclosed.

The properties obtained using the cohesionless aggregate from the two tests (i.e. φ values of 56.1° and) 57.7° are comparable. Therefore, based on the result of these tests it was concluded that the disclosed devices are accurate and can be used to determine the shear strength properties of aggregates and concrete.

### Example 2

A device and methods as disclosed herein was used to evaluate the characteristics of PCPC mixtures.

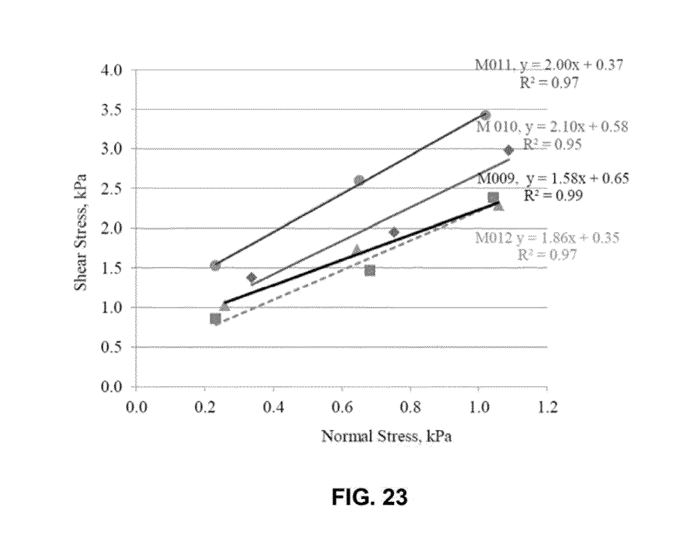

FIG. 23 shows the plot of normal stress vs. shear stress for selected PCPC mixtures. The regression equations are also provided with the plots. The coefficient of internal friction and the force of cohesion were determined as described herein from the information.

The coefficient of internal friction and the force of cohesion were used to calculate the spreading force (pulling force) required for pervious concrete. The models, fitted for the coefficient internal friction, force of cohesion and pulling force are presented in the following equations:

Force of cohesion (kPa)=0.68 V+1.19 P+0.80 A−0.1.23 VP−0.85 VA−1.29 PA

Internal Friction=1.79V+2.80 P+1.59 A−1.21 PA

Pulling Force (kg)=43.9 V+30 P+46 A−49 PA
wherein: V=the ratio of the volume of voids to the total volume

P=the ratio of the volume of paste to the total volume

A=the ratio of the total volume of aggregate to the total volume

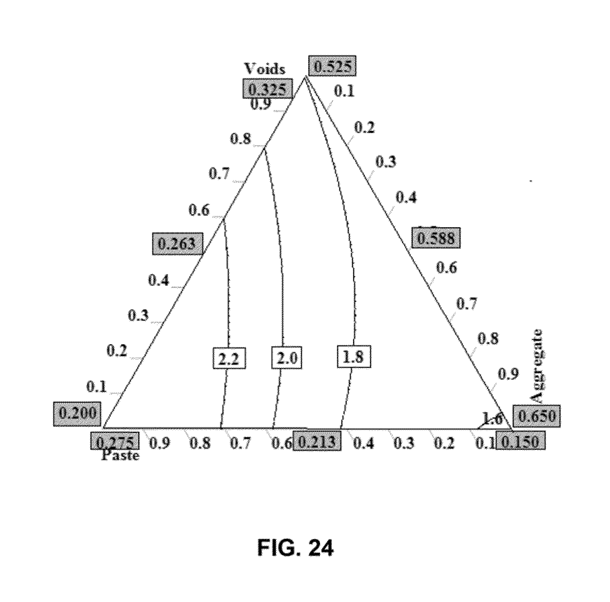

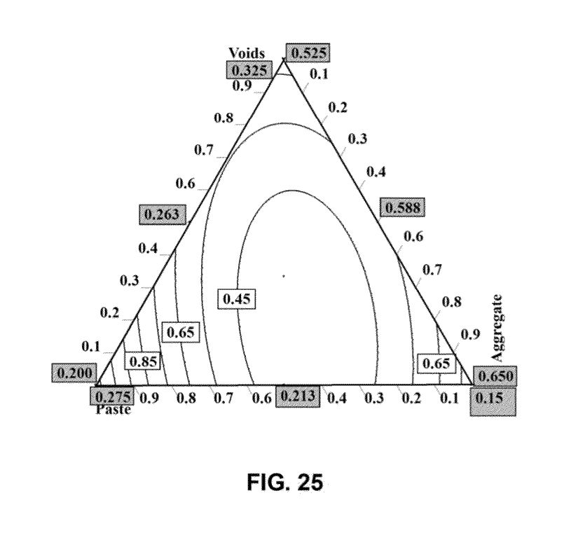

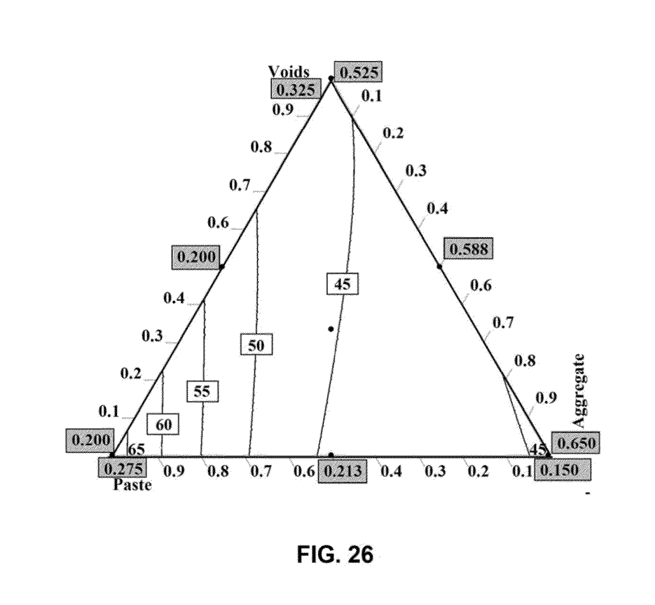

The contour plot of the coefficient internal friction (FIG. 24) and force of cohesion (FIG. 25) was developed based on the models. A contour plot of the pulling force is shown in FIG. 26. The effect of paste volume on pulling force seemed significant. As the paste volume increased the required pulling force also increased significantly. The increase in pulling force was also associated with the increase in internal shear resistance of the mix. The effect of paste volume on the coefficient of internal friction and the force of cohesion was significant.

The quadratic model fitted for coefficient of internal friction had a good predicting power (R²=0.92). The model for force of cohesion also had fair predicting power (R²=0.78). Pulling force had a fair coefficient of regression (R²=0.77) and good lack of fit f-statistics (Prob>F=0.85). Thus, the model can be acceptable for making predictions.

While the present subject matter has been described in detail with respect to specific embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A shear testing device comprising:
a base, the base comprising a containment frame and defining a receiving area, the containment frame defining a first opening at an upper surface of the containment frame, the base comprising a first end that is closer to the containment frame than to the receiving area;
a sample container, the sample container defining a second opening at a lower surface of the sample container, the sample container being slidably mountable on the base such that at a first position, the first opening and the second opening are aligned and form a shear plane at an interface of the containment frame and the sample container; and
a hoist in mechanical communication with the first end of the base.

2. The shear testing device of claim 1, wherein the base further comprises a base frame, the containment frame being removably attachable to the base frame.

3. The shear testing device of claim 1, further comprising a lift point indicator.

4. The shear testing device of claim 3, the lift point indicator indicating an angle of lift of the first end.

5. The shear testing device of claim 1, further comprising a receiver for receiving a ballast load.

6. The shear testing device of claim 1, the sample container comprising one or more frictionless bearings.

7. The shear testing device of claim 1, wherein the hoist is a screw hoist.

8. A method for determining one or more shear strength parameters of a granular material comprising:
aligning a first opening of a containment frame of a base of a shear testing device with a second opening of a sample container such that the containment frame and the sample container together define a sample loading area and define a shear plane at an interface of the containment frame and the sample container, the sample container being slidably mounted on the base;
loading a granular material sample into the sample loading area;
lifting a first end of the base to a lift point at which the sample container slides from a first position to a second position due to gravitational force acting on the sample container, the base being at an angle to horizontal at the lift point; and
determining one or more shear strength parameters of the granular material from the angle.

9. The method of claim 8, wherein the granular material is concrete.

10. The method of claim 9, wherein the granular material is pervious concrete.

11. The method of claim 8, further comprising applying a ballast load to the sample container.

12. The method of claim 8, further comprising determining at least one of the weight and the volume of the granular sample that is confined to the sample container.

13. The method of claim 8, wherein the granular material sample is about 10 kilograms or less.

14. The method of claim 8, wherein the one or more shear strength parameters comprise shear stress at failure and normal stress at failure.

15. The method of claim 8, wherein the one or more shear strength parameters comprise the coefficient of internal friction or the cohesion.

16. The method of claim 8, wherein the one or more shear strength parameters comprises the pulling force.
